(19) United States
(12) Patent Application Publication
(10) Pub. No.: US 2003/0054007 A1
(43) Pub. Date: Mar. 20, 2003

(54) Intracellular protein delivery compositions and methods of use

(76) Inventors: Philip L. Felgner, Rancho Santa Fe, CA (US); Olivier Zelphati, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/738,046

(22) Filed: Dec. 15, 2000

Related U.S. Application Data
(60) Provisional application No. 60/172,441, filed on Dec. 17, 1999.

(51) Int. Cl. A61K 39/395; A61K 48/00

(52) U.S. Cl. 424/178.1; 514/8

(57) ABSTRACT

The present invention relates to compositions and methods for intracellular protein delivery. The compositions include a protein operatively associated with a cationic lipid in such a way as to facilitate intracellular delivery of the protein by the cationic lipid, such as by associating directly with a cationic lipid, encapsulating it in a cationic liposome, associating the protein with a lipoplex comprising cationic lipid and nucleic acid, or associating the protein with an anionic polymer that is in association with a cationic lipid. These compositions are useful in delivering antibodies to intracellular proteins to neutralize their activity, and to introduce therapeutically useful proteins, peptides or small molecules.

### RELATED APPLICATION

This application claims priority under 35 U.S.C. §119(e) to U. S. Provisional Application Serial No. 60/172,441, filed Dec. 17, 1999.

### FIELD OF THE INVENTION

The present invention relates to compositions and methods for delivery of functional proteins into living cells.

### DESCRIPTION OF THE RELATED ART

During the last 15 years, there has been considerable progress toward the development of increasingly effective transfection reagents for the delivery of transcriptionally active DNA into cultured cells (Felgner et al., Proc. Natl. Acad. Sci. U.S.A. 84: 7413-7417, 1987; Felgner et al, J. Biol. Chem. 269: 2550-2561, 1994; Zelphati et al., Pharm. Res. 13: 1367-1372, 1996). In addition, there has been a growing understanding of the mechanistic aspects of nucleic acid delivery within synthetic delivery systems (Zelphati et al., Proc. Natl. Acad. Sci. U.S.A. 93: 11493-11498, 1996; Tseng et al., J. Biol. Chem. 272: 25641-25647, 1997). In particular, cationic lipids have been shown to be very effective agents for the delivery of nucleic acid into cells and there are numerous commercial reagents available for this purpose, including Lipofectin™ and LipofectAMINE™ (Gibco BRL). Plasmid transfection using such reagents is now a routine laboratory procedure commonly used in most biomedical laboratories. Procedures for preparing liposomes for transfection formulations are described in U.S. Pat. Nos. 5,264,618 and 5,459,127, and by Felgner et al. (Proc. Natl. Acad. Sci. U.S.A. 84: 7413-7417, 1987).

Surprisingly, there has been much less progress towards identifying reagents to deliver functional recombinant proteins into cells. This is in spite of considerable effort in biotechnology companies and academic laboratories devoted to producing recombinant proteins and monoclonal antibodies. Historically, the drug discovery programs at most pharmaceutical companies have been directed toward extracellular targets, i.e. either cell surface receptors or proteins produced and secreted by cells. As a result, currently approved therapeutic proteins for the treatment of various diseases are all secretory proteins (representative examples shown in Table 1; for a recent review of proteins and antibodies currently approved for human use, see, Glennie and Johnson, Immunol. Today 21: 403-410 [2000]; Reichert, Trends Biotechnol. 18: 364-369 [2000]). The ongoing programs at the leading genomics companies are also directed at identifying human genes encoding secretory or membrane proteins as potential candidates for drug development. If there were effective methods for introducing proteins into cells, it would not be necessary to restrict the potential therapeutic candidates to secretory and membrane proteins and there would be an even larger pool of recombinant proteins that could be considered as potential drug candidates.

TABLE 1

| Product (company) | Protein |
|---|---|
| Humulin (Lilly) | Insulin |
| Intron A (Biogen) | Interferon-α |
| Avonex (Biogen) | Interferon-β |
| Epogen (Amgen) | Erythropoietin |
| Infergen (Amgen) | Interferon |
| Neupogen (Amgen) | Granulocyte colony stimulating factor |
| Activase (Genentech) | Tissue-plasminogen activator |
| Nutropin/Protropin (Genentech) | Growth hormone |
| Pulmozyme (Genentech) | DNase |
| Herceptin (Genentech) | Anti-Her2 recombinant antibody |
| Remicade (Centocor) | Anti-TNF antibody |
| Rituxan (IDEC/Genentech) | Anti-CD20 recombinant antibody |

The direct delivery of monoclonal antibodies into viable cells could be used to specifically inhibit intracellular targets. In fact, some investigators have used DNA transfection to introduce antibodies into cells. Intracellular Antibodies (“Intrabodies”) are single-chain antibodies derived from a parent monoclonal antibody in which the variable domains of the light and heavy chains are joined together by a flexible peptide linker. The resulting recombinant gene product retains the ability of the parent antibody to bind to and neutralize the target antigen. The entire intrabody sequence can be encoded on an expression plasmid, and the plasmid can be transfected into cultured cells leading to intracellular expression of the intrabody protein and neutralization of its intracellular protein antigen. The effects of intrabodies have been investigated using structural, regulatory, and enzymatic proteins of the human immunodeficiency virus (HIV-1) as targets (Mhashilkar, et al., EMBO J. 14: 1542-1551 [1995]; Mhashilkar, et al, J. Virol. 71: 6486-6494 [1997]; Mhashilkar, et al., Hum. Gene Ther. 10: 1453-1467 [1999]). Although the use of intrabodies is conceptually attractive, the method is time consuming and labor intensive. Moreover, monoclonal antibody proteins are much easier to obtain. If a reagent could be developed that could efficiently deliver proteins into cells, it would make research with monoclonal antibodies directed against intracellular antigens much more convenient. Some of the same intracellular targets that have been demonstrated through the use of intrabodies should be accessible with a reagent that directly delivers the antibody into the cell.

Another approach for delivering proteins into cells that has recently received some attention, uses “protein transduction domains” (PTDs), such as the third helix of the Drosophila Antennapedia homeobox gene (Antp), the HIV Tat, and the herpes virus VP22, all of which contain positively charged domains enriched for arginine and lysine residues (Schwarze, et al., Trends Cell Biol. 10: 290-295 [2000]; Schwarze, et al., Science 285: 1569-1572 [1999]). In some cases hydrophobic peptides derived from the signal sequences have been used successfully for the same purpose (Rojas, et al., J. Biol. Chem. 271: 27456-27461 [1996]; Rojas, et al., Nature Biotechnol. 16: 370-375 [1998]; Du, et al., J. Pept. Res. 51: 235-243 [1998]). Coupling of these peptides to marker proteins such as β-galactosidase has been shown to confer efficient internalization of the marker protein into cells. More recently, chimeric, in-frame fusion proteins containing these PTDs have been used to deliver proteins to a wide spectrum of cell types both in vitro and in vivo. However, this approach requires an additional step of conjugation which may adversely affect biological activity of the protein. For example, it may distort the conformation of the protein or may sterically interfere with the function of the protein.

As is apparent from the foregoing discussion, there is a need to develop a convenient and reliable reagent that can deliver proteins, peptides and antibodies into cells. The ability to directly inhibit or initiate targeted intracellular functions specifically in live cells by the delivery of antibodies or recombinant proteins will be of tremendous benefit in all aspects of cellular biology and functional genomics. A general methodology that can be employed with numerous cell types and under a wide range of conditions would certainly contribute significantly to the analysis of complex phenotypes. Ultimately the application of effective reagents of this kind, which accomplish intracellular recombinant protein and monoclonal antibody delivery, would contribute to the discovery and development of new therapeutic modalities directed against various diseases such as cancer, inflammatory disorders, and infectious diseases. For example, the functional delivery of factors controlling transcription could potentially regulate the uncontrolled proliferation of cells characteristic of conditions such as cancer and inflammatory diseases. Similarly, overexpression of specific genes, such as those encoding growth factors, growth factor receptors, cytokines and regulatory proteins involved in signal transduction could be controlled by the intracellular delivery of proteins regulating transcription. Conversely, conditions with under-expression of critical genes, such as tumor suppressors and growth factors, could be rectified by intracellular delivery of the relevant proteins.

The present invention addresses the need for intracellular protein delivery by providing convenient, reproducible reagents for this purpose which can be quickly prepared for delivery of any desired protein. These reagents may be optimized and reformulated to deliver proteins into cells in vivo as a therapeutic treatment for a variety of diseases.

### SUMMARY OF THE INVENTION

One embodiment of the present invention is a composition for intracellular delivery of a protein, comprising a protein in operative association with a cationic intracellular delivery vehicle comprising a cationic lipid, wherein intracellular delivery vehicle is adapted to fuse with a cell membrane, thereby effecting intracellular delivery of associated protein. Preferably, the protein is linked, either directly or through a linker, to a cationic lipid. This can be done, for example, by linking protein to a polynucleotide, and associating polynucleotide with a cationic lipid, such as through a PNA linker or through a linker molecule that is linked to a cationic lipid. One preferred linker molecule is malemide. Preferably, at least one of the protein-linker and linker-cationic lipid links is covalent. Optionally, at least one of the protein-linker and linker-cationic lipid links is ionic.

Regardless of the composition of the delivery vehicle, the protein can advantageously be a therapeutic protein, a diagnostic protein, an antigenic protein, a prophylactic protein (e.g., a vaccine), a polyclonal antibody, a monoclonal antibody, an antibody fragment or engineered antibody, or another specific binding protein. In one embodiment of the invention, the intracellular delivery vehicle comprises a cationic lipid, a cationic liposome, a lipoplex comprising cationic lipid and nucleic acid, or an anionic polymer in association with a cationic lipid. When the delivery vehicle comprises an anionic polymer in association with a cationic lipid, the anionic polymer may include a reactive group coupled to the protein. In one embodiment, the anionic polymer is a biopolymer, such as nucleic acid. In another embodiment, the polymer is a synthetic polymer.

The present invention also includes a method for delivering a protein to a cell, comprising providing protein associated with a cationic lipid in such a manner as to form an intracellular delivery composition, and contacting the delivery composition with a cell membrane of a cell, such that the cationic lipid forms an association with cell membrane and thereby delivers protein into cell. Typically, the cationic lipid will fuse with the cell membrane, thereby allowing the associated protein to enter the cell. The present method can be used with any of the delivery compositions, proteins, or vehicles contemplated herein. Thus, in one embodiment, the delivery composition includes a nucleic acid. Preferably the nucleic acid is attached to the protein, either directly, or indirectly through a linker such as a PNA. In another embodiment, the delivery composition a cationic liposome encapsulating the protein, or a cationic lipid linked to the protein through a covalent linker. In some embodiments, the protein inhibits an intracellular process, or the protein is therapeutic, or the protein is an antibody or antibody fragment.

Other particular embodiments of the invention include a protein or peptide delivery composition comprising a protein or peptide encapsulated by a cationic liposome. In one embodiment the cationic lipid is XG40. The cationic liposome optionally includes a co-lipid. Suitable co-lipids include dioleoylphosphatidyl ethanolamine (DOPE), polyethyleneglycol-phosphatidylethanolamine (PEG-PE), diphytanoyl-PE, cholesterol and monooleoylglycerol.

The invention also includes a method of forming a protein or peptide encapsulated by a cationic liposome, comprising step of mixing a dried cationic lipid film and a protein or peptide solution.

Further, the invention includes method for delivering a protein or peptide into a cell, comprising steps of providing a cationic liposome-encapsulated protein or peptide formed by mixing a solution of the protein or peptide with a dried cationic lipid film; and contacting the cell with the cationic liposome-encapsulated protein.

Another embodiment of the invention is a protein or peptide delivery composition, comprising a polynucleotide, a peptide nucleic acid (PNA) bound to the polynucleotide, wherein the PNA includes a reactive chemical group capable of binding to a protein, a protein bound to the reactive chemical group, and a cationic lipid.

In some embodiments of the invention, the protein is an antibody, antibody fragment, or other specific binding molecule that inhibits a step in a metabolic pathway, or that binds to an intracellular antigen.

Yet another aspect of the present invention is a method for delivering a protein or peptide into a cell, comprising step of contacting the cell with a composition comprising a polynucleotide, a peptide nucleic acid (PNA) bound to the polynucleotide, wherein the PNA includes a reactive chemical group capable of binding to a protein, a protein bound to the reactive chemical group, and a cationic lipid.

Still another embodiment is a protein or peptide delivery composition, comprising a protein, a negatively charged polymer having a reactive chemical group capable of coupling to the protein, and a cationic liposome which interacts with the negatively charged polymer. The negatively charged polymer can be an oligonucleotide, for example.

The present invention includes a method of making a protein or peptide delivery composition, comprising step of combining a protein, a negatively charged polymer having a reactive chemical group capable of coupling to the protein, and a cationic liposome which interacts with the negatively charged polymer.

It also includes a method for delivering a protein or peptide into a cell, comprising step of contacting the cell with a composition comprising a protein, a negatively charged polymer having a reactive chemical group capable of coupling to the protein, and a cationic liposome which interacts with the negatively charged polymer.

Still another aspect of the invention is a protein or peptide delivery composition, comprising a cationic liposome, wherein the liposome includes a reactive chemical group capable of binding to a protein, and a protein bound to the reactive chemical group. Maleimide is one example of a suitable reactive chemical group.

The invention may be embodied in a method for delivering a protein or peptide into a cell, comprising step of contacting the cell with a composition comprising a cationic liposome, wherein the liposome includes a reactive chemical group capable of binding to a protein, and a protein bound to the reactive chemical group.

### BRIEF DESCRIPTION OF THE DRAWINGS

FIG. 1 is a schematic diagram showing the formation of the first protein delivery reagent (Reagent I) from dried cationic lipid film and a monoclonal antibody, binding of the encapsulated antibody to the plasma membrane, and intracellular delivery of the encapsulated antibody.

FIG. 2 is a schematic diagram of the second protein delivery reagent (Reagent II). A maleimide-labeled peptide nucleic acid (PNA) clamp is combined with a plasmid to generate a maleimide-labeled plasmid. A reduced antibody is then combined with the maleimide-labeled plasmid which is transfected into cells using conventional DNA transfection reagents.

FIG. 3 is a schematic diagram of pGeneGrip™ vector showing a PNA clamp bound to a PNA binding site on the plasmid.

FIG. 4 is a schematic diagram of a method for producing streptavidin-labeled plasmid DNA using a biotin-labeled PNA clamp.

FIG. 5 is a schematic diagram of a third protein delivery reagent (Reagent III). An activated oligonucleotide is bound to a monoclonal antibody to form an antibody/oligonucleotide conjugate which is then combined with a cationic liposome. The complex is then transfected into cells using conventional DNA transfection reagents.

FIG. 6 is a schematic diagram of a fourth protein delivery reagent (Reagent IV). A bifunctional cross-linking reagent such as SPDP is used to conjugate a protein of interest with a maleimide activated cationic lipid. The mixture is then added onto cells leading to cellular uptake of the protein liposome conjugate.

FIG. 7 shows Reagent I mediated delivery of various proteins into Jurkat cells and induction of apoptosis. The histograms show FACS analysis of cells that were treated with either a BSA-phycoerythrin conjugate (BSA-PE), or a mixture of BSA-PE and either granzyme-B, caspase-3, cytochrome-c or caspase-8. The y-axis on these histograms quantifies the amount of the fluorescent BSA-phycoerythrin that enters the cells, and the x-axis quantifies the amount of apoptosis using CaspaTag assay.

### DETAILED DESCRIPTION OF THE PREFERRED EMBODIMENTS

Unless defined otherwise, technical and scientific terms used herein have the same meaning as commonly understood by one of ordinary skill in the art to which this invention belongs. See, e.g. Singleton, et al., Dictionary of Microbiology and Molecular Biology 2nd ed., J. Wiley & Sons (New York, N.Y. 1994); Sambrook, et al., Molecular Cloning, A Laboratory Manual, Cold Springs Harbor Press (Cold Springs Harbor, N.Y., 1989); Ausbel, et al., Current Protocols in Molecular Biology, Volume 1 and 2, Greene Publishing Association and Wiley-Interscience, New York, 1991; Harlow and Lane, Antibodies, A Laboratory Manual, Cold Springs Harbor Press (Cold Springs Harbor, N.Y., 1999). One skilled in the art will recognize many methods and materials similar or equivalent to those described herein, which could be used in the practice of the present invention. It is to be understood that this invention is not limited to particular methodology described. The terminology used herein is for the purpose of describing particular embodiments only, and is not intended to be limiting, since the scope of the present invention will be limited only by the appended claims.

The present invention provides convenient and reproducible reagents for the delivery of proteins, peptides and other small molecules into cultured cells that are as effective and convenient to use as are DNA transfection reagents. These reagents allow the role of intracellular recombinant proteins affecting signaling pathways, regulating the cell cycle, controlling apoptosis, determining oncogenesis, and regulating transcription to be directly assessed intracellularly. The reagents can also be used for the in vitro or in vivo delivery of antibodies or peptides which block the function of specific intracellular proteins and affect cellular metabolism, cell viability or virus replication. For example, antibodies to transcription factors which promote transcription of undesirable genes can be used to inhibit the activity of these proteins. These protein delivery reagents will facilitate the identification of therapeutically useful monoclonal antibodies and recombinant proteins directed against intracellular targets and affecting intracellular metabolic pathways.

The ability to directly inhibit targeted intracellular functions specifically in live cells will be of tremendous benefit in all aspects of cellular biology and functional genomics. Furthermore, a general methodology that can be employed with numerous cell types and under a wide range of conditions would contribute significantly to the analysis of complex phenotypes. Formulation of new antibiotics by designing antibodies targeting specific viral or bacterial products could also help in the control of infectious diseases.

Four classes of intracellular protein delivery reagents are disclosed in the present invention: Reagents I-IV. These four reagent configurations are discussed below.

In a preferred embodiment, for all four reagents, a second lipid called a co-lipid or helper lipid is included in the cationic lipid formulation. Although DOPE (dioleoylphosphatidylethanolamine) is the most widely used helper lipid, other neutral lipid molecules such as polyethylene glycol-phosphatidylethanolamine (PEG-PE), diphytanoyl-PE, cholesterol and monooleoylglycerol can also be used.

The reagents disclosed herein can be used to deliver any protein of interest, including therapeutically useful proteins (e.g. tumor suppressor proteins, cystic fibrosis transmembrane regulator (CFTR), adenosine deaminase (ADA), hexoseaminidase A, peptides, wild type protein counterparts of mutant proteins and cell surface receptors such as those for cytokines (e.g. interleukins, interferons, colony stimulating factors) and peptide hormones.

### Preparation of Protein Delivery Reagents

Reagent I

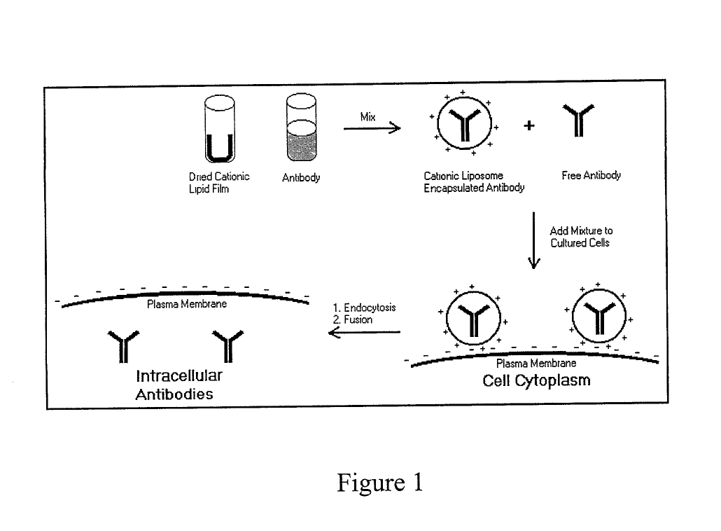

The first protein delivery reagent (Reagent I) takes advantage of the surprising result that cationic lipid formulations can deliver antibodies into cells by mixing the antibody solution with a dried cationic lipid (FIG. 1). The procedure involves suspending a dried cationic lipid film with a solution of the protein to be delivered. During this lipid hydration step, liposomes form and some of the protein that is dissolved in the hydration medium becomes encapsulated in the liposomes. The majority of the protein is present in the free (unencapsulated) form. The mixture is added to cultured cells, or introduced in vivo, and the cationic liposomes containing encapsulated protein attach to negatively charged cell surfaces. Following cell surface attachment, the liposomes fuse directly with the plasma membrane and deliver their encapsulated protein into the cell (FIG. 1). Alternatively, the liposomes can be endocytosed and then fuse with the endosome, releasing the liposome encapsulated protein into the cytoplasm. The efficacy of this procedure depends on the lipid composition of the liposomes.

The ability of a particular cationic liposome-encapsulated protein to deliver the protein into cells can be easily determined by one of ordinary skill in the art using the methods described herein. The cationic lipid films used to make Reagent I comprise various amounts of cationic lipid and, preferably, a co-lipid such as dioleoylphosphatidylethanolamine (DOPE). Cationic lipids for use in the present invention include, for example, those described in U.S. Pat. Nos. 4,897,355, 5,264,618 and 5,459,127, the entire contents of which are incorporated herein by reference. One particularly preferred cationic lipid composition, called XG40, is described in co-pending application Ser. No. 09/448,876, filed Nov. 24, 1999, entitled “Amphiphilic Polyamide Compounds”, the entire contents of which are incorporated herein by reference. The structure and synthesis of XG40 is described below. Suitable co-lipids comprise, but are not limited to lysophosphatides, phosphatidylethanolamines, phosphatidylcholines, cholesterol derivatives, fatty acids, mono-, di- and tri-glyceride phospholipids having a neutral headgroup (Liu, et al., Nature Biotech. 15: 167-173 [1997]; Hong, et al., FEBS Lett. 400: 233-237 [1997]). Other suitable single-chain lyso lipids comprise the Rosenthal inhibitor ester and ether derivatives disclosed in U.S. Pat. Nos. 5,264,618 and 5,459,127 to Felgner, et al., the entire contents of which are hereby incorporated by reference.

In the experiments described herein, the cationic lipid composition of Reagent I comprises XG40 and the co-lipid DOPE

Synthesis of 18-1-Lys-5Tc

Step 1: To a solution containing 10.4 gram (20 mmol) of dioctylamine in 100 ml CH₂Cl₂:methanol (1:1), 50 ml acrylonitrile was added. The mixture was briefly heated to 60° C. and cooled to room temperature for 12 hours. The solvent and the excess acrylonitrile were removed by a rotovapor followed by high vacuum. The solid was dissolved in hexane and subjected to normal phase silica gel chromatographic purification. The resulting N-propylnitrile-N-dioctadecylamine was dissolved in 100 ml dioxane and cooled to 4° C. and then reduced to N-propylamine-dioctadecylamine (18-1) (see reaction scheme) using LiAlH₄. Excess LiAlH₄ was neutralized with dilute NaOH. The organic phase was filtered, diluted with CH₂Cl₂ and washed with water. High yield of 18-1 as white solid was recovered and air dried with Na₂SO₄, evaporation of solvents and dried under high vacuum. The resulting 18-1 was used in the next step without further purification.

Step 2: To a solution of 18-1 containing 1:1 ratio of triethylamine (TEA), di-Boc-lysine NHS ester was added at 1:2:1 ration to the amine. After reaction for 2 h at room temperature, the resulting di-boc-lysine amide of 18-1 was purified using silica gel. Deprotection of di-boc-lysine with TFA/CH₂Cl₂ resulted in 18-1-lys-1. After routine work-up and removal of solvent, the 18-1-lysine amine was generated in high yield and used in the next reaction without further purification.

Step 3: To a solution of 18-1-lysine-1 in CH₂Cl₂ with TEA, α-CBZ-ε-Boc-Lysine, previously acetylated with dicyclohexylcarbodiimide (DCC) and N-hydroxylsuccinamide (NHS) was added at 2:4:1 ratio to the lysine amide. The reaction was monitored with Nihhydrin reaction until its completion. The resulting bis (Z-Boc-lysys) lysine amide was purified with silica gel after work-up. The Boc group was removed and bis-Zlys3-18-1 was used for the next reaction.

Step 4: B is Z-boc-lysyl (Bis(Z)lys-3-18-1) and was obtained similar to step 3 and purified by silica gel similar to Z-Boc-lysyl lysine amide. Deprotection of the intermediate with TFA/CH₂Cl₂ removed the Boc groups. Further deprotection with Pd/H₂ in EtOH resulted in the final product 18-1-lys 5Tε.

Chemical Structure of 18-1-Lys-5Tε

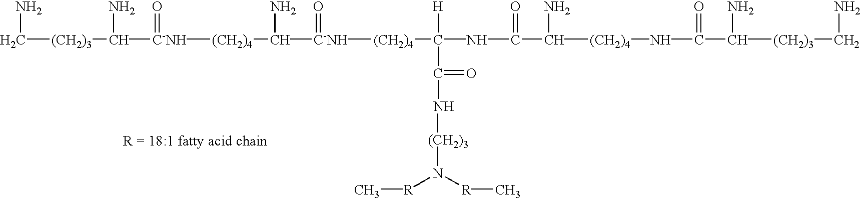

Synthesis of Trifluoroacetylated Derivatives of 18-1-Lys-5 Tε

Acetate or trifluoroacetate groups were conjugated to the deprotected primary amino groups in the polar headgroup by reacting the purified product with acetic acid or trifluoroacetic acid. Synthesis of randomly trifluoroacetylated derivatives of XG40 is illustrated below: The N-hydroxysuccinamide ester of trifluoroacetic acid (TFA) was prepared from TFA, N-hydroxysuccinamide and dicyclohexylcarbodiimide (DCCI) in dimethylformamide (DMF) in a molar ratio of 1:1.1: 1.1, incubated for 20 minutes at room temperature and was then filtered to remove the dicyclohexylurea. One gram of XG40 was dissolved in 10 ml dry methanol and a 10 molar excess of triethylamine (TEA) was added. The activated TFA was added to the XG40 in an appropriate molar ratio to give the desired level of trifluoroacetylation and the mixture was incubated at room temperature for 2 hours. The solvent was evaporated under vacuum, re-dissolved in 5 ml methanol and precipitated with 200 ml ether at −70 C. The precipitate was collected by centrifugation and the process was repeated once. The product was dissolved into 5 ml dry methanol and converted into the methanesulfonic acid (MeS) salt form by reaction with 2 molar excess of MeS to amino groups. The excess of MeS was removed by repeat ether precipitation. The final product was dried under vacuum as white powder.

Preparation of Cationic Liposomes

Cationic lipid films were prepared by mixing organic (preferably chloroform) solutions of the lipid in type I borosilicate glass vials and removing the organic solvent by evaporation under ambient conditions, preferably in a sterile hood. Vials were placed under vacuum overnight to remove solvent traces. To produce cationic liposomes, an appropriate amount of sterile pyrogen-free water or other aqueous vehicle was added, and the vials were vortexed at the top speed for 1-2 minutes at room temperature. To screen a particular cationic lipid compound, various solvents were evaluated to ensure that both the cationic lipid and co-lipid (if present) remained soluble during the evaporation step. For example, a solvent mixture of 80% chloroform and 20% methanol may be used. The lipid solution was then dried, resulting in a uniform lipid film. The drying step may be performed in several ways, including evaporation in a rotary evaporator, evaporation under ambient conditions, or blow drying under a stream of nitrogen gas. Procedures for preparing liposomes for transfection formulation are disclosed and exemplified in the '618 and '127 patents mentioned above. Other procedures for liposome formulation are disclosed in Felgner, et al., Proc. Natl. Acad. Sci. USA 84: 7413-7417 (1987).

Reagent II

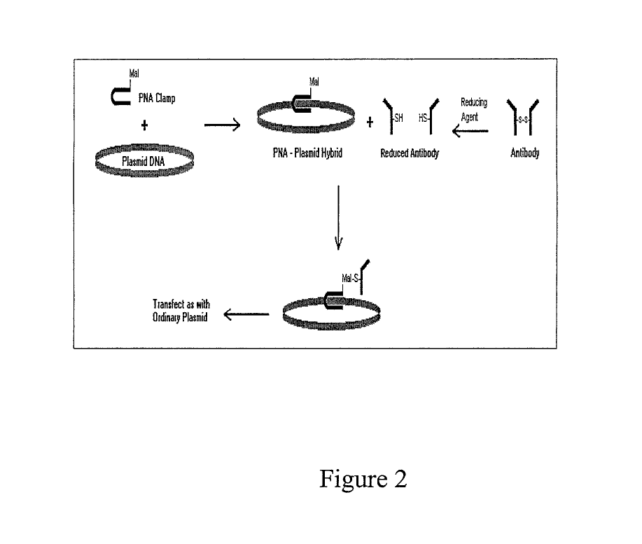

The second protein delivery reagent (Reagent II) involves attaching the protein of interest to a polynucleotide (DNA or RNA), preferably a plasmid, and transfecting the plasmid into cells with a conventional DNA transfection reagent (FIG. 2). These types of complexes are called lipoplexes because proteins become captured in a nucleic acid-cationic lipid complex. The term “lipoplex” was defined in order to distinguish between the encapsulation that occurs with ordinary liposomes and a different type of organization that occurs when cationic lipid based transfection reagents are mixed with DNA (Felgner et al., Hum. Gene Ther. 8: 511-512, 1997). Both the DNA and the cationic liposomes rearrange and compact together forming a complex called a “lipoplex.” One hundred % of the DNA is captured into the cationic lipid-DNA lipoplex. The lipoplex does not have an internal fluid volume as do the liposomes. When lipoplexes are properly formulated, they can form virus-like particles that can deliver functional DNA into cultured cells in vitro and into tissues in vivo. The DNA used in this method may be linear double-stranded DNA, linear single-stranded DNA, circular double-stranded DNA or circular single-stranded DNA.

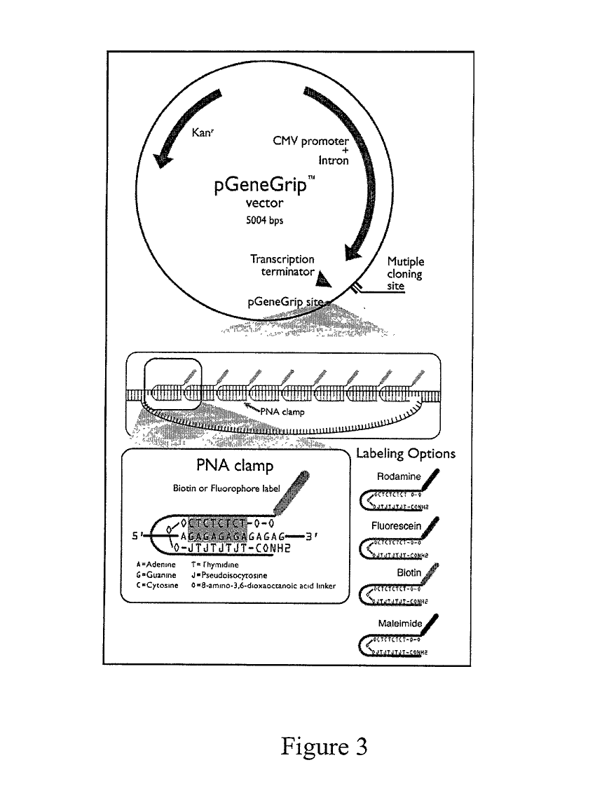

In one embodiment, peptide nucleic acid (PNA) clamping technology is used to attach proteins to plasmid DNA. PNA clamps may be used to attach various ligands, including proteins and peptides, onto DNA. This technology is called “PNA dependent gene chemistry” (PDGC) and is described by Zelphati, et al., BioTechniques 28: 304-310 (2000), in PCT WO98/19503, and in co-pending U.S. patent application Ser. No. 09/224,818, the entire contents of which are incorporated herein by reference. PNA is a polynucleotide analog that has the deoxyribose-phosphate backbone of DNA replaced by a peptide backbone (FIG. 3). The PNA clamp hybridizes with its complementary binding site on a plasmid to form a highly stable PNA-DNA-PNA triplex clamp.

A plasmid, pGeneGrip™, is available from Gene Therapy Systems, Inc. (San Diego, Calif.) that contains PNA binding sites as shown in FIG. 3. Several different labeled PNA clamps can be used, including PNA labeled with biotin, reactive chemical groups such as maleimide, and fluorescent labels such as rhodamine and fluorescein. An 80 base pair polypurine -AG- repeat sequence (pGeneGrip site) was cloned after the terminator of a cytomegalovirus (CMV) immediate early gene promoter-based plasmid. This region of the plasmid was selected for insertion of the binding site because it is not involved in transcription and PNA binding to this region does not affect expression (Zelphati et al., Hum. Gene Ther. 10: 15-24, 1999). A complementary PNA clamp was synthesized consisting of an 8 base -CT- repeat, a 3 unit flexible linker (8-amino-3,6-dioxaoctanoic acid), and an 8 base -JT-repeat, where J is pseudoisocytosine, an analog of C, which encourages formation of the Hoogsteen triplex hybrid (Zelphati et al., 1999, supra.; Egholm et al., Nucl. Acids Res. 23: 217-222, 1995). The -CT- stretch hybridizes to the -AG- repeat on the plasmid in an anti-parallel Watson-Crick manner, and the -JT-stretch binds in the major groove of the PNA-DNA hybrid via Hoogsteen interactions to form the PNA-DNA-PNA triplex clamp (Egholm et al., supra.). The non-target DNA strand is displaced, forming the non-hybridized “D-loop” (Bukanov et al., Proc. Natl. Acad. Sci. U.S.A. 95: 5516-5520, 1998; Cherny et al., Proc. Natl. Acad. Sci. U.S.A. 90: 1667-1670, 1993).

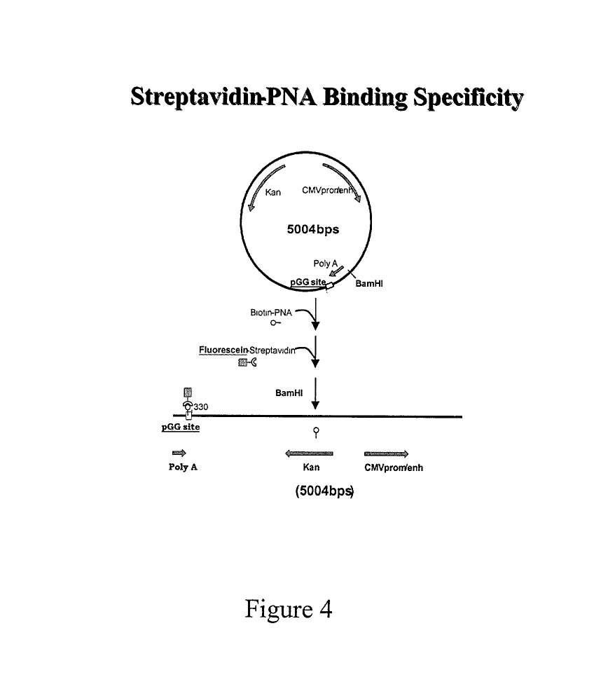

In one embodiment, the biotin-streptavidin system is used to couple proteins to DNA. Streptavidin is captured by a DNA-PNA-biotin hybrid. Several well known chemical methods for covalently attaching peptides and proteins to streptavidin can be used. For example, any ligand that contains a free sulfhydryl group will react with streptavidin that contains a conjugated maleimide moiety. Peptide-streptavidin conjugates are added directly to biotin-PNA-DNA. The preparation of streptavidin labeled plasmid DNA is shown in FIG. 4. First, biotin-PNA was added to the pGeneGrip™ and the unbound biotin-PNA was removed by ethanol precipitation. Streptavidin was added to the biotin-PNA labeled plasmid and this product was purified by gel filtration to remove unbound streptavidin. Quantitative analysis of the gel filtration data showed that there was about one bound streptavidin for every plasmid. After streptavidin labeling, the plasmid, which contains a single BamHI site 310 base pairs from the PNA binding site, was restricted with the Bam HI enzyme. Cryo-atomic force microscopy images of the streptavidin labeled DNA revealed linearized DNA and a white dot on each strand showing the location of the streptavidin molecule. Virtually every strand had a single streptavidin positioned precisely 310 base pairs away from the end of each strand at the predicted location of the PNA binding site. These results illustrate the exquisite specificity and selectivity of this approach for labeling plasmid DNA, and also show that the PNA approach can be used to attach proteins onto DNA. This method is suitable for intracellular delivery of any protein or peptide.

Reagent III

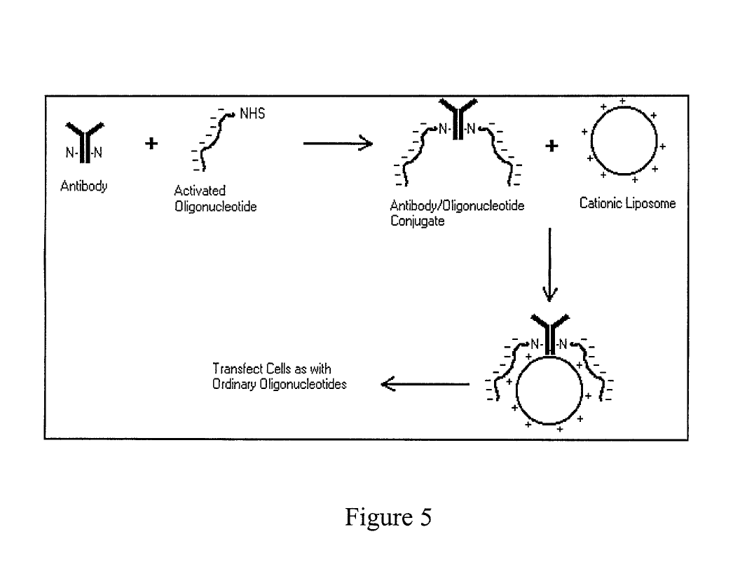

The third protein delivery reagent (Reagent III) involves attachment of polynucleotides (DNA or RNA), preferably oligonucleotides, to a protein using established conjugation chemistry, followed by the use of conventional cationic lipid transfection reagents. The DNA may be linear double-stranded DNA, linear single-stranded DNA, circular double-stranded DNA or circular single-stranded DNA. The concept of protein delivery using Reagent III is illustrated in FIG. 5. Although an oligonucleotide and an antibody are shown in the illustration, the method is suitable for delivery of any protein or peptide into a cell. In addition, the method is not limited to the use of a polynucleotide. Any negatively charged biologically compatible polymer capable of interacting with a cationic liposome is within the scope of the present invention. These polymers include, for example, heparin, dextran sulfate, polyglutamic acid etc. The oligonucleotide is activated by attaching a chemical group capable of reacting with a protein to be delivered to a cell by standard methods. In one embodiment, an oligonucleotide is conjugated to available amino groups on the protein of interest by using NHS-activated oligonucleotide (FIG. 5). The protein is added to a vial containing dry NHS-activated oligonucleotide and the resulting protein oligonucleotide conjugate is purified from the unreacted oligonucleotide using a Sephadex G-50 spin column. The resulting protein oligonucleotide conjugate is then transfected into cells using a conventional cationic lipid transfection reagent. Another method for coupling oligonucleotides to proteins is described below.

Activation of Oligonucleotides

Various reactive chemical groups can be attached to oligonucleotides or other negatively charged polymers, or to PNA molecules, using methods well known in the art. A variety of crosslinking agents can be used to target different chemical groups on proteins, including amino, carboxyl, sulfhydryl, aryl, hydroxyl and carbohydrates. Many of these crosslinking reagents are available from Pierce Chemical Co. (Rockford, Ill.) and described in the Pierce catalog. Heterobifunctional crosslinkers contain two or more different reactive groups that allow for sequential conjugations with specific groups of proteins, minimizing undesirable polymerization or self-conjugation. Heterobifunctional crosslinkers which react with primary or secondary amines include imidoesters and N-hydroxysuccinimide (NHS)-esters such as SMCC and succimidyl-4-(p-maleimidophenyl)-butyrate (SMPB). Cross-linkers which react with sulfhydryl groups include maleimides, haloacetyls and pyridyl disulfides. Carbodiimide cross-linkers couple carboxyls to primary amines or hydrazides, resulting in formation of amide or hydrazone bonds. One widely used carbodiimide cross-linker is 1-ethyl-3-(3-dimethylaminopropyl)-carbodiimide (EDC) hydrochloride.

For example, maleimide-labeled PNA is obtained by reacting PNA with SMCC, and pyridyldithiol-labeled PNA is obtained by reacting PNA with ([N-succinimidyl 3-(2-pyridyldithio)propionate) (SPDP). Both of these groups react with protein sulfhydryl groups. Any desired chemical group can be conjugated to PNA using conventional chemical methods.

Coupling of Oligonucleotides to Proteins

An FITC or Rhodamine-linked, amine-modified oligonucleotide was dissolved in 0.1 M sodium borate, 2 mM EDTA, pH 8.25, at a concentration of 9 nmol in 15 μl. Disuccinimidyl suberate (DSS) was dissolved in dry dimethylsulfoxide (DMSO) at a concentration of 1 mg/100 μl (prepared fresh). Sixty μl of the DSS solution was added to the oligonucleotide and the solution was mixed well and incubated for 15 min at room temperature in the dark. The solution was vortexed vigorously and centrifuged at 15,000 rpm for 1 min to separate the two phases. The upper layer was carefully removed and discarded. The extraction was performed two more times with n-butanol. The remaining samples were chilled on dry ice and lyophilized for 15-30 min to remove the last traces of liquid. Four hundred ,g of goat IgG (Sigma Chemical Co., St. Louis, Miss.) was dissolved in 0.1 M sodium phosphate, 0.15 M NaCl, pH 7.2, at a concentration of 20 mg/ml. Ten μl of the IgG solution was added to the dried, DSS-activated oligonucleotide, mixed gently to dissolve and reacted for 1 hour at room temperature in the dark. Unconjugated oligonucleotide was removed using a Sephadex G-75 spin column (3 min at 3,000 rpm). The conjugate was transfected using a conventional cationic lipid transfection reagent.

Reagent IV

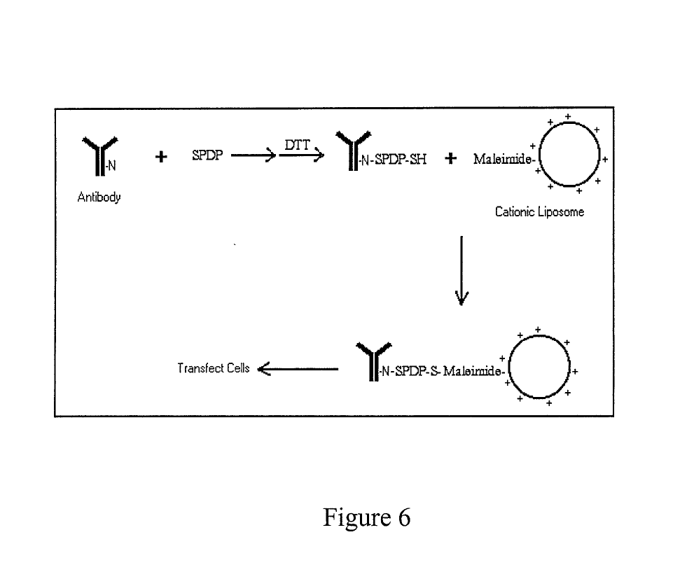

Another way to get proteins to associate with cationic liposomes is by chemical conjugation of the protein to a lipid that is incorporated in the cationic liposome as shown in FIG. 6. There are many methods available in the art for coupling hydrophobic moieties onto peptides or proteins. Detailed methods have been compiled in several books including “Bioconjugate Techniques”, Greg T Hermanson, Academic Press Inc. or “Liposome Technology”, volume I, II and III, 2^{nd} edition, G. Gregoriadis, CRC press. Various reactive chemical groups can be attached to the lipid and/or proteins using methods well known in the art, as described above for Reagent II in the activation of oligonucleotide section. A variety of crosslinking agents including heterobifunctional crosslinkers (SPDP, SMPB, NHS, SATA, SMCC, etc) can be used to target different chemical groups on proteins and/or lipids, including amino, carboxyl, sulfhydryl, aryl, hydroxyl and carbohydrates. Many of these crosslinking reagents are available from Pierce Chemical Co. (Rockford, Ill.) and described in the Pierce catalog.

One approach involves the use of the amine reactive reagent N-succinimidyl 3-(2-pyridyldithio) propionate (SPDP). SPDP was incubated with an antibody such that a SPDP/protein mole ratio of 5:1 was obtained after a 20 min incubation at room temperature. The product was isolated by gel filtration prior to the sample being reduced with dithiothreitol to generate a reactive —SH group. The thiolated product was isolated by gel filtration. Coupling of the thiolated antibody to liposomes was preformed by incubating thiolated antibody at room temperature with the maleimide-liposomes at a ratio of 75 μg protein per 750 μg of lipid. The coupling of SPDP to lipid was done according to published procedures (e.g., see Hermanson, supra; Leserman and Barbet, Nature 288: 602-604 [1980]). Coupling of amine modified antibodies typically resulted in 15-25 μg antibody/750 μg of lipid.

### Pharmaceutical Compositions

One aspect of the present invention relates to administration of the delivery compositions disclosed herein directly to cells, in vitro. In another embodiment of the invention, the compositions are delivered to cells in vivo. In particular, the compositions may be used to deliver protein intracellularly in almost any type of animal cell, including birds, fish, mammals, and amphibians. The mammals treated with proteins according to the present invention can be non-human or human. Any of the proteins currently known or later discovered to have therapeutic value can be used in the invention. Further, proteins specifically affecting intracellular processes are particularly suitable for the present invention. The present invention is not limited to nor does it focus on any particular protein; rather, the focus is on particular methods and compositions suitable for delivering any protein into a cell.

Pharmaceutically acceptable compositions contemplated for use in the practice of the present invention can be used in the form of a solid, a solution, an emulsion, a dispersion, a micelle, a liposome, and the like, wherein the resulting composition contains one or more of the active compounds contemplated for use herein, as active ingredients thereof, in admixture with an organic or inorganic carrier or excipient suitable for nasal, enteral or parenteral applications. The active ingredients may be compounded, for example, with the usual non-toxic, pharmaceutically or physiologically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use. The carriers that can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents may be used. The active compounds contemplated for use herein are included in the pharmaceutical composition in an amount sufficient to produce the desired effect upon the target process, condition or disease.

In addition, such compositions may contain one or more agents selected from flavoring agents (such as peppermint, oil of wintergreen or cherry), coloring agents, preserving agents, and the like, in order to provide pharmaceutically elegant and palatable preparations. Tablets containing the active ingredients in admixture with non-toxic pharmaceutically acceptable excipients may also be manufactured by known methods. The excipients used may be, for example, (1) inert diluents, such as calcium carbonate, lactose, calcium phosphate, sodium phosphate, and the like; (2) granulating and disintegrating agents, such as corn starch, potato starch, alginic acid, and the like; (3) binding agents, such as gum tragacanth, corn starch, gelatin, acacia, and the like; and (4) lubricating agents, such as magnesium stearate, stearic acid, talc, and the like. The tablets may be uncoated or they may be coated by known techniques to delay disintegration and absorption in the gastrointestinal tract, thereby providing sustained action over a longer period. For example, a time delay material such as glyceryl monostearate or glyceryl distearate may be employed. The tablets may also be coated by the techniques described in the U.S. Pat. Nos. 4,256,108; 4,160,452; and 4,265,874, incorporated herein by reference, to form osmotic therapeutic tablets for controlled release.

When formulations for oral use are in the form of hard gelatin capsules, the active ingredients may be mixed with an inert solid diluent, for example, calcium carbonate, calcium phosphate, kaolin, or the like. They may also be in the form of soft gelatin capsules wherein the active ingredients are mixed with water or an oil medium, for example, peanut oil, liquid paraffin, olive oil and the like.

Of course, oral formulations may need suitable protection from gastric processes, and may be in the form of buffered compositions, time release compositions, enteric-coated compositions, and the like, as is well known in the art. It will be appreciated that not all proteins can be effectively delivered through the oral route, and that certain other routes discussed herein may also be unsuitable for particular protein delivery compositions.

Formulations may also be in the form of a sterile injectable suspension. Such a suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,4-butanediol. Sterile, fixed oils are conventionally employed as a solvent or suspending medium. For this purpose, any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Formulations contemplated for use in the practice of the present invention may also be administered in the form of suppositories for rectal administration of the active ingredients. These compositions may be prepared by mixing the active ingredients with a suitable non-irritating excipient, such as cocoa butter, synthetic glyceride esters of polyethylene glycols (which are solid at ordinary temperatures, but liquify and/or dissolve in the rectal cavity to release the active ingredients), and the like.

In addition, sustained release systems, including semi-permeable polymer matrices in the form of shaped articles (e.g., films or microcapsules) can also be used for the administration of the protein delivery reagents of the present invention.

The amount of the protein delivery compositions of the invention administered to a vertebrate, preferably a mammal (e.g., dogs, cats, primates, horses, sheep, cows), more preferably a human, will vary depending upon the condition to be treated, the severity of the condition, and the response of the patient to the treatment. In general, the amount administered is between about 0.01 μg/kg and 1,000 mg/kg, preferably between about 0.1 μg/kg and 100 mg/kg, and more preferably between about 1 μg/kg and 10 mg/kg. Dosage optimization can be performed using standard dose-response curves known to one of ordinary skill in the art.

The present invention also includes the preparation of a medicament for treatment of a human or animal, wherein the medicament is for intracellular delivery of a protein and wherein it comprises a formulation of the type described herein. In one aspect of the invention, the medicament is for the treatment of a disease having an intracellular component. The medicament can be for treating disease by inhibiting or facilitating an intracellular process. The focus of the present invention is broader than treatment of any particular disease; rather, the focus is on treatment of a wide variety of conditions affecting or affected by an intracellular process that could benefit from intracellular delivery of a protein.

### EXAMPLES

The following examples are offered by way of illustration and not by way of limitation. The examples are described so as to provide those of ordinary skill in the art with a complete disclosure and description of how to make and use the compounds, compositions, and methods of the invention, and are not intended to limit the scope of what the inventors regard as their invention.

### Example 1

### Use of Reagent I for Intracellular Delivery of Proteins

For cationic lipid-mediated DNA delivery (i.e. lipofection), the lipids are usually suspended in water to form liposomes before they are added to the DNA. The positively charged liposomes interact spontaneously with negatively charged DNA and essentially 100% of the DNA forms cationic lipid-DNA complexes called lipoplexes. The positively charged lipoplex, which carries the entrapped DNA, interacts with negatively charged cell surfaces, and through a series of steps the entrapped DNA enters the cytoplasm and ultimately enters the nucleus where it can be transcribed.

Standard lipofection technology relies on the interaction between highly positively charged liposomes and negatively charged DNA. Since proteins do not share the same physical properties as DNA, this technology has not yet been directly applied to protein delivery. Because proteins do not have the same high negative charge density as DNA, lipoplex formation does not occur spontaneously. Furthermore, different proteins interact very differently with cationic liposomes depending on whether they have a net positive or negative charge. Since the charge characteristics of different proteins vary widely, it has been difficult to prepare a general protein delivery reagent using this approach.

Certain amphipathic lipid molecules spontaneously organize themselves into bilayer membranes when suspended in water (Gregoriadis et al., FEBS Lett. 402: 107-110 [1997]; Gregoriadis et al., Methods 19: 156-162, 1999). Solubilizing the lipids into an organic solvent, followed by evaporation of the solvent, produces a dried film consisting of an amorphous mixture of these bilayer-forming lipids. Addition of water to the dried lipid film causes the lipids to spontaneously organize into bilayers, which, upon shaking, form closed vesicles called liposomes that contain an internal volume. A fraction of the solutes present in the hydration buffer are encapsulated during liposome formation; however, the bulk of the solute remains unencapsulated. The physical behavior of liposomes has been well studied and they have been investigated extensively as potential drug delivery vehicles. There are several approved human clinical products which take advantage of liposome drug encapsulation ability.

The protein delivery reagent I of the present invention incorporates proteins into cationic liposomes by encapsulation. Several fluorescent antibodies were used to demonstrate the utility of Reagent I for delivery of protein cargo. An FITC-labeled monoclonal antibody against a telomere repeat-binding factor-2 (TRF-2) was obtained from ImGeneX (San Diego, Calif.) and FITC-labeled goat IgG and anti-actin antibodies were purchased from Sigma. For visualization of fluorescent antibody uptake by cells, the day before the experiment, NIH3T3 cells were seeded onto 22 mm coverslips so that they were 50-90% confluent on the day of the experiment. XG40 cationic lipid (1.224 mg) and 0.254 mg DOPE were dissolved in 750 μl chloroform. The XG40/DOPE mixture (2.5 μl) containing approximately 5 μg of lipid was dispensed in a polypropylene tube. The chloroform was removed under a stream of nitrogen. A fluorescently-labeled antibody was diluted in 10 mM HEPES, pH 7, 150 mM NaCl (HBS) at 10-160 μg/ml, preferably 80-100 μg/ml. The diluted antibody was added to the dried film, and the solution was vortexed immediately at medium speed for 10 seconds. Serum-free medium was added to the tube to make up the final volume to 200 μl. The coverslips were blotted dry and placed in a 35 mm petri dish. The cationic lipid/antibody complexes were transferred onto the cells which were incubated at 37° C. and 5% CO₂ for 4 hours or longer. Additional growth medium was added if longer incubation time was desired. Antibody uptake was visualized by fluorescence microscopy.

Background fluorescence was determined using NIH-3T3 cells incubated with the fluorescein labeled antibody alone, without Reagent I. Reagent I greatly enhanced the uptake of the fluorescein labeled antibody in NIH-3T3 cells compared to the very small amount of uptake that occurred with the antibody alone. Some of the antibody in the cells appeared to be uniformly distributed in the cytoplasm, some was concentrated at the cell surface, and there were some brightly fluorescent aggregates apparently bound to the cell surface. Most of the nuclei were darker than the cytoplasm, suggesting that the antibody was being excluded from the nuclei. Treatment of cells with Reagent I did not appear toxic based on the appearance of the cells. There was no apparent reduction in fluorescence intensity even after 24 hours.

Similar results were obtained using Jurkat, HeLa S3, BHK-21, CHO-K1, B16-F0 and 293 cells, although there were differences between the cell types in the fluorescence intensity, percentage of positive cells and the patterns of intracellular fluorescence. The background fluorescence in the absence of Reagent I for all the cell types was the same as for NIH-3T3. Two different monoclonal antibodies directed against different antigens and obtained from different commercial suppliers gave essentially the same results, illustrating that this method is generally applicable to intracellular protein delivery.

The ability of Reagent I to deliver a wide variety of proteins was further examined. For this purpose, FITC-labeled high and low molecular weight dextran, goat IgG and anti-actin antibody were used. Reagent I was found to deliver all the proteins intracellularly. Low molecular weight dextran (10,000 MW) was able to enter into the nucleus of the transduced cells, whereas, high molecular weight dextran (70,000 MW) did not enter the nucleus. Goat IgG and anti-actin antibody were also excluded from the nucleus of the transduced cells. Anti-actin antibody showed some evidence of accumulating onto intracellular actin filaments, however not surprisingly, the staining pattern is different from that observed on fixed and permeabilized cells.

In order to examine the retention of biological activity of proteins introduced into the cells, Reagent I was used to deliver caspase-3 (generous gift from Dr. Guy Salvensen), cytochrome-c (Sigma), granzyme-B (CalBiochem) and caspase-8 (Biovision) into Jurkat cells and the induction of apoptosis was monitored. Cells were seeded in a 24-well plate at a cell density of 0.5×10⁶ cells per well. The different proteins were diluted in PBS at 40-160 μg/ml. Then, caspase 3 (100 nmoles), caspase 8 (1 unit), cytochrome c (100 μg/ml) and granzyme B (200 units) solutions were used to hydrate the Reagent I as described earlier. The cationic lipid/protein complexes were transferred onto the cells which were incubated at 37° C. and 5% CO₂ for 4 hours or longer. Additional growth medium was added if longer incubation time was desired. Cells were transduced with either BSA-phycoerythrin (BSA-PE) alone or BSA-PE along with caspase-3, cytochrome-c, granzyme-B or caspase-8. Flow cytometry was used to monitor fluorescence of BSA-phycoerythrin (BSA-PE) as a measure of protein uptake, whereas the extent of apoptosis induction was monitored by CaspaTag assay as a measure of functional activity of the introduced proteins. CaspaTag Fluorescein Caspase Activity kit was purchased from Intergen (N.Y.). Briefly, 300 μl of cells were transferred into a fresh tube and 30 ×Working Dilution FAM-VAD-FMK (10 μl) was added to the cell suspension. The cells were mixed by slightly flicking the tubes and incubated for one hour under 5% CO₂ and protected from light. Then 2 ml of 1×Working Dilution Wash Buffer was added to the labeling mix and cells were spun down at 400×g for 5 minutes at room temperature. The supernatant was discarded and the cell pellet was gently vortexed to disrupt cell clumps. Cell pellet was re-suspended in 1×Working Dilution Wash Buffer and samples were analyzed by FACS.

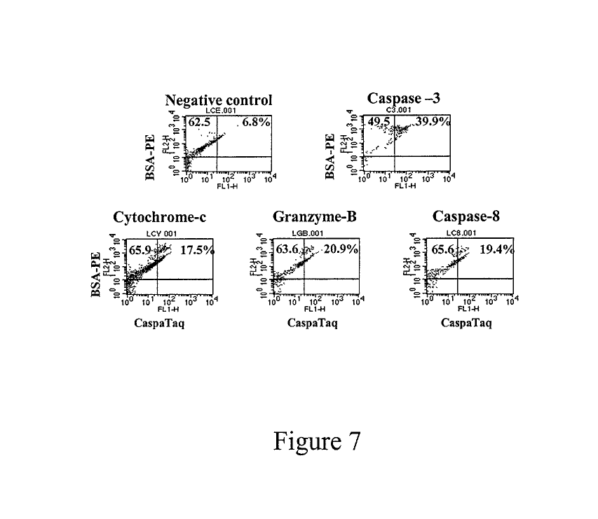

As shown in FIG. 7, caspase-3 was the most potent apoptosis inducer leading to induction of apoptosis in about 40% of the cells. Cytochrome-c, granzyme-B and caspase-8 gave rise to about 20% apoptotic cells, whereas the background level was about 7%. The results demonstrate that Reagent I not only aids in the intracellular delivery of proteins, but also preserves the functional integrity of the delivered proteins. Thus, protein delivery reagent described herein can be used for functional delivery of any protein.

### Example 2

### Intracellular Delivery of Streptavidin Using Reagent II

Colloidal gold (10 nm diameter)-labeled streptavidin (Sigma, St. Louis, Mo.) was mixed with biotin-PNA labeled plasmid at 10:1 molar ratio excess and incubated for 1 hour at 37° C. The mixture was then passed over a Sephacryl-500-HR column to remove the free streptavidin-gold and the gold labeled plasmid was transfected into COS 7 cells with the GenePORTER™ (Gene Therapy Systems, Inc., San Diego, Calif.) transfection reagent. The results showed that streptavidin-gold labeled plasmid can be transfected into cells with streptavidin-gold still attached. The intracellular plasmid in the transfected cells was revealed by transmission electron microscopy. The results also showed that streptavidin-gold can be delivered into cells by binding the streptavidin onto the plasmid. Streptavidin-gold-labeled plasmid DNA was seen in the extracellular space and in the cytoplasm. Gold particles were also found attached to the cell surface and in endocytic vesicles.

In another PDGC approach, the maleimide moiety was conjugated directly to the PNA. The N-hydroxysuccinimide (NHS) ester end of the succimidyl 4-(N-maleimidomethyl) cyclohexane-1-carboxylate (SMCC) was first reacted with the 5′ primary amine of the PNA clamp to form a stable amide bond. Then, the maleimide-PNA conjugate was hybridized to its binding site on the plasmid. Plasmid DNA containing the PNA binding site was incubated with SMCC-PNA to allow hybridization, and the mixture was ethanol precipitated to remove free PNA. The nuclear localization signal peptide containing a terminal cysteine residue was reduced and mixed with maleimide-PNA labeled plasmid. The mixture was purified by ethanol precipitation and examined by agarose gel electrophoresis. The results showed that the plasmid containing reactive maleimide became labeled with the NLS peptide, but a plasmid containing biotin-PNA was not labeled. These results demonstrate that peptide conjugation is dependent on the reaction between the reduced sulfhydryl group and maleimide moiety on the PNA. DNA/PNA fluorescein was used as a control to show where the DNA migrated into the gel.

In another embodiment, a plasmid containing maleimide-labeled PNA (Gene Therapy Systems) was used. Partially reduced fluorescein isothiocyanate (FITC)-labeled antibody was prepared by adding 3 mg of 2-mercaptoethylamine to 250 μg of protein in 0.5 ml phosphate buffered saline (PBS), pH 7.4. The mixture was incubated for 90 minutes at 37° C., and the reduced antibody was purified by gel filtration chromatography on a Sephadex G-25 column to remove excess reducing agent. The reduced antibody was coupled to the maleimide-PNA labeled plasmid by incubating 2 moles of antibody per mole of plasmid at 37° C. for 90 minutes, and the product was used directly in transfection assays without further purification (FIG. 2). Alternatively, unreacted antibody can be removed from the antibody-plasmid conjugate by Sephacryl 500 HR column chromatograpy. The plasmid/protein conjugate was then transfected into cells using conventional DNA transfection reagents and protocols. Intracellular fluorescence revealed successful uptake of the labeled antibodies by the cells.

In the case of antibodies, the free sulfhydryl group can be exposed by reduction with 2-mercaptoethylamine, the excess reducing agent can be removed by Sephadex G-50 column chromatography, and the resulting reduced antibody can be added to the maleimide labeled plasmid to produce the DNA-antibody conjugate, which can be transfected into cells with the transfection reagent.

### Example 3

### Intracellular Delivery of Proteins Using Reagent III

An oligonucleotide obtained from a commercial supplier (GenBase, Inc.) containing a 5′ terminal NH2 group and a 3′ terminal Rhodamine moiety (5′-NH2-TGACTGTGAACGTTCGAGATGA-Rhodamine-3′) was conjugated to goat IgG (Sigma) and was introduced into cells using a conventional cationic lipid transfection reagent. Two variations of the method were tested. In one, lipid formulation was first resuspended in hydration buffer to form liposomes and then antibody-oligonucleotide conjugate was added to the liposome formulation. This approach leads to the formation of lipoplexes. In another variation, antibody-oligonucleotide conjugate was directly added to the dried film of BioPORTER reagent. This approach leads to encapsulation of the protein-oligonucleotide conjugates as well as lipoplex formation. Either approach was found to be successful in the intracellular delivery of antibody-oligonucleotide conjugates.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of that which is described and claimed.

What is claimed is:

1. A composition for intracellular delivery of a protein, comprising:
a protein in operative association with a cationic intracellular delivery vehicle comprising a cationic lipid, wherein the intracellular delivery vehicle is adapted to fuse with a cell membrane, thereby effecting intracellular delivery of the associated protein.

2. The composition of claim 1, wherein the protein is linked, either directly or through a linker, to a cationic lipid.

3. The composition of claim 2, wherein the protein is linked to a cationic lipid by linking the protein to a polynucleotide, and associating the polynucleotide with a cationic lipid.

4. The composition of claim 3, wherein the protein is linked to the polynucleotide through a PNA linker.

5. The composition of claim 1, wherein the protein is linked to a linker molecule that is linked to a cationic lipid.

6. The composition of claim 5, wherein the linker molecule is maleimide.

7. The composition of claim 5, wherein at least one of the protein-linker and linker-cationic lipid links is covalent.

8. The composition of claim 5, wherein at least one of the protein-linker and linker-cationic lipid links is ionic.

9. The composition of claim 1, wherein the protein is a therapeutic protein.

10. The composition of claim 1, wherein the protein is a specific binding protein.

11. The composition of claim 10, wherein the specific binding protein is an antibody or antibody fragment.

12. The composition of claim 1, wherein the intracellular delivery vehicle comprises a cationic lipid, a cationic liposome, a lipoplex comprising cationic lipid and nucleic acid, or an anionic polymer in association with a cationic lipid.

13. The composition of claim 12, wherein the intracellular delivery vehicle comprises an anionic polymer in association with a cationic lipid, and wherein the anionic polymer includes a reactive group coupled to said protein.

14. A method for delivering a protein to a cell, comprising:
providing the protein associated with a cationic lipid in such a manner as to form an intracellular delivery composition, and
contacting the delivery composition with a cell membrane of a cell, such that the cationic lipid forms an association with the cell membrane and thereby delivers the protein into the cell.

15. The method of claim 14, wherein the delivery composition is the composition of claim 1.

16. The method of claim 14, wherein the delivery composition further includes a nucleic acid

17. The method of claim 16, wherein the nucleic acid is attached to the protein.

18. The method of claim 16, wherein the nucleic acid is linked to the protein through a PNA.

19. The method of claim 14, wherein the delivery composition comprises a cationic liposome encapsulating the protein.

20. The method of claim 14, wherein the delivery composition comprises a cationic lipid linked to the protein through a covalent linker.

21. The method of claim 14, wherein the protein inhibits an intracellular process.

22. The method of claim 14, wherein the protein is therapeutic.

23. The method of claim 14, wherein the protein is an antibody or antibody fragment.
